(19) United States
(12) Patent Application Publication
(10) Pub. No.: US 2002/0001840 A1
(43) Pub. Date: Jan. 3, 2002

(54) Methods and compositions for modulating integrin-mediated cell-cell interactions

(76) Inventors: Carlos Lopez-Otin, Asturias (ES); Jose Maria Perez Freije, Asturias (ES); Santiago Cal Miguel, Asturias (ES); Jose Manuel Lopez Garcia, Asturias (ES); Albert Bernard Bianchi, Princeton, NJ (US); Pamela Trail, Madison, CT (US)

Correspondence Address: Joan E. Switzer, Bristol-Myers Squibb Company, P.O. Box 4000, Princeton, NJ, 08543-4000, US

(21) Appl. No.: 09/824,129

(22) Filed: Apr. 2, 2001

Related U.S. Application Data
(60) Provisional application No. 60/194,164, filed on Apr. 3, 2000.

(51) Int. Cl. C12N 5/06; C07H 21/04; A61K 38/43; C12N 9/00

(52) U.S. Cl. 435/325; 435/183; 424/94.1; 536/23.2; 530/350

(57) ABSTRACT

Compositions and methods are provided for identifying and designing modulators of integrin-mediated cell-cell interactions through altering the interaction of ADAM 23 with αvβ3 integrin. Compositions and methods are also provided for modulating integrin-mediated cell-cell interactions such as those involved in angiogenesis, induction of active metalloproteinases, tumor progression and neural tissue growth.

This invention claims the benefit of priority of U.S. Provisional Application Serial No. 60/194,164, filed Apr. 3, 2000.

This invention was supported in part by funds from the U.S. government (NIH Grant Nos. GM47157 and GM49899). The U.S. government may therefore have certain rights in the invention.

### FIELD OF THE INVENTION

The present invention relates to compositions and methods for identifying modulators of the interaction of a disintegrin and metalloproteinase domain, referred to herein as ADAM 23, with αvβ3 integrin via use of these compositions. The present invention also relates to methods of using the identified agents to modulate the interaction of ADAM 23 with αvβ3 integrin. Modulators of integrin-mediated cell-cell interactions relating to the interaction of ADAM 23 with αvβ3 integrin are expected to be useful therapeutically in various applications including, but not limited to, altering tumor progression, and in particular angiogenesis and induction of active matrix metalloproteinases facilitating migration of tumor cells, and in modulating growth of neural tissue.

### BACKGROUND OF THE INVENTION

Cell-cell and cell-extracellular matrix interactions are essential for the development and maintenance of an organism as well as for the progression of malignant tumors. Likewise, proteolysis of the extracellular matrix is of vital importance for a series of tissue-remodeling processes occurring during both normal and pathological conditions, such as tissue morphogenesis, wound healing, inflammation, and tumor cell invasion and metastasis. These events are mediated by a variety of cell surface adhesion proteins and proteases with different structural and functional characteristics (Werb, Z. Cell 1997 91:439-442). Among them, a group of recently described proteins called ADAMs (a disintegrin and metalloproteinase domain) have raised considerable interest due to their potential ability to perform both functions, adhesion and proteolysis (Wolfsberg et al. J. Cell. Biol. 1995 131:275-278; Blobel, C. P. Cell 1997 90:589-592; Wolfsberg, T. G. and White, J. M. Dev. Biol. 1997 180:389-401). These membrane proteins have a unique domain organization containing pro-, metalloproteinase-like, disintegrin-like, cysteine-rich, EGF-like, transmembrane, and cytoplasmic domains. Some of these domains are similar to those found in a family of soluble snake venom proteins that bind with high affinity to the platelet integrin GPIIb/IIIa, inhibiting platelet aggregation, and causing hemorrhage in snake bite victims (Niewiarowski et al. Semin. Hematol. 1994 31:289-300).

ADAMs, also known as cellular disintegrins or MDCs (metalloprotease, disintegrin, and cysteine-rich domains), have been found in a wide variety of mammalian tissues as well as in other eukaryotic organisms including Xenopus laevis (Alfandari et al. Dev. Biol. 1997 182:314-330; Cai et al. Dev. Biol. 1998 204:508-524), Drosophila melanogaster (Rooke et al. Nature 1996 273:1227-1231), and Caenorhabditis elegans (Podbilewicz, B. Mo. Biol. Cell 1996 7:1877-1893). Members of this protein family were first associated with reproductive processes; however, in recent years the family has widely expanded and to date, more than 20 different ADAMs with diverse functions have been identified and characterized at the molecular level. Thus, in addition to a series of family members such as fertilins or cyritestins, involved in spermatogenesis and heterotypic sperm-egg binding and fusion (Blobel et al. Nature 1992 356:248-252; Houliva et al. Curr. Opin. Cell Biol. 1996 8:692-699; Adham et al. DNA Cell Biol. 1998 17:161-168), other ADAMs like meltrin α, are implicated in homotypic myoblast-myoblast fusion (Yagami-Hiromasa et al. Nature 1995 377:652-656; Gilpin et al. J. Biol. Chem. 1998 273:157-166). Meltrin α and meltrin β have also been suggested to play a role in osteoblast differentiation and/or osteoblast activity in bone (Inoue et al. J. Biol. Chem. 1998 273:4180-4187). The cellular disintegrins MS2 (ADAM 8) and decysin have been identified as monocytic and dendritic cell-specific proteins, suggesting that they may be involved in host defense mechanisms (Yoshida et al. Int. Immunol. 1990 2:585-591; Mueller et al. J. Exp. Med. 1997 189:655-663). Similarly, ADAMTS-1, characterized by the presence of thrombospondin motifs in its amino acid sequence, has been associated with various inflammatory processes (Kuno et al. J. Biol. Chem. 1997 272:556-562). ADAMTS-4, another member of this subfamily of disintegrins containing thrombospondin motifs, has been characterized as an aggrecanase responsible for the degradation of cartilage aggrecan in arthritic diseases (Tortorella et al. Science 1999 284:1664-1666). Other ADAMs have been found to function as proteolytic enzymes involved in the processing of relevant cellular substrates. For example, TACE (TNF-α converting enzyme) is an ADAM implicated in the release of proinflammatory membrane anchored cytokine TNF-α from the plasma membrane (Black et al. Nature 1997 385:729-733; Moss et al. Nature 1997 385:733-736). The product of the kuz gene from Drosophila (ADAM 10), also appears to be responsible for proteolytic activation of the transmembrane protein Notch required for lateral inhibitory signaling during neurogenic differentiation (Pan, D. and Rubin, G. M. Cell 1997 90:271-280; Sotillos et al. Development 1997 124:4769-4779). Other studies have proposed that Kuz is required for processing of the Notch ligand Delta (Qi et al. Science 1999 283:91-94). MDC9/ADAM 9 has been reported to be involved in the ectodomain shedding of membrane-anchored heparin-binding EGF-like growth factor (Izumi et al. EMBO J. 1998 17:7260-7272).

In addition to this variety of physiological functions described for ADAMs, some of these family members have been suggested to be involved in the development and progression of tumor processes. For example, ADAM 11 was originally identified as a candidate tumor suppressor gene for human breast cancer (Emi et al. Nat. Genet. 1993 5:151-157) and ADAMTS-1 has been associated with the development of cancer cachexia (Kuno et al. J. Biol. Chem. 1997 272:556-562). Several disintegrins have also been associated with pathological features of hematological malignancies including the premature egression of leukemic cells from bone marrow into the peripheral blood or the generalized connective tissue destruction accompanying these malignant processes (Wu et al. Biochem. Biophys. Res. Commun. 1997 235:437-442). In addition, ADAM 10 has been found to be overexpressed in tumors of sympathoadrenal origin such as pheochromocytomas and neuroblastomas (Yavari et al. Hum. Mol. Genet. 1998 7:1161-1167). Other ADAM family members with proteolytic activity such as TACE have been proposed to play indirect roles in tumor processes through their participation in the proteolytic activation and release of membrane-bound cytokine or growth factor precursors of relevance in cancer (Black et al. Nature 1997 385:729-733; Moss et al. Nature 1997 385:733-736).

### SUMMARY OF THE INVENTION

An object of the present invention is to provide an isolated nucleic acid sequence encoding ADAM 23, a new member of the disintegrin family of proteins. Also provided are vectors containing this nucleic acid sequence and host cells transfected with these vectors which express ADAM 23.

Another object of the present invention is to provide methods for identifying agents which alter integrin-mediated cell-cell interactions through modulating the interaction of ADAM 23 with αvβ3 integrin.

Another object of the present invention is to provide synthetic peptides comprising the amino acid sequence AVNECDIT (SEQ ID NO:1) which modulate the interaction of ADAM 23 with αvβ3 integrin. Host cells expressing such peptides are also provided.

Another object of the present invention is to provide methods of designing modulators of the interaction of ADAM 23 with αvβ3 integrin.

Another object of the present invention is to provide methods of altering integrin-mediated cell-cell interactions which comprise contacting cells with a modulator of the interaction of ADAM 23 with αvβ3 integrin. Integrin-mediated cell-cell interactions which can be modulated via these agents include, but are not limited to, angiogenesis and induction of active matrix metalloproteinases facilitating migration of tumor cells and growth of neural tissue.

Another object of the present invention is to provide methods for inhibiting tumor progression in a patient which comprise administering to the patient a modulator of the interaction of ADAM 23 with αvβ3 integrin.

Another object of the present invention is to provide a method for inducing neural tissue growth which comprises contacting neural tissue with a modulator of the interaction of ADAM 23 with αvβ3 integrin.

Yet another object of the present invention is to provide pharmaceutical compositions comprising a modulator which alters the interaction αvβ3 integrin with ADAM 23 and a pharmaceutically acceptable vehicle.

### BRIEF DESCRIPTION OF THE FIGURES

FIG. 1 is a bar graph showing the activities of various proteins including basic fibroblast growth factor (bFGF), recombinant ADAM 23 disintegrin domain (dd) expressed as a GST fusion protein (GST-ADAM23dd) or glutathione (GST) at varying concentrations in a murine MATRIGEL plug angiogenesis model. MATRIGEL containing either bFGF, GST-ADAM23dd at 4, 20.5 or 61.5 μg/plug, or GST at 4, 20.5 or 61.5 μg/plug were implanted subcutaneously in female athymic mice. On day 7, MATRIGEL plugs were harvested and the number of cells in each plug section was determined using a video imaging system.

FIG. 2 shows microscopic views at either 10× or 20× of sections of paraffin-embedded, hematoxylin-eosin stained MATRIGEL plugs harvested on day 7 from female athymic mice. Microscopic views of MATRIGEL plugs containing vascular growth factor (VEGF) and basic fibroblast growth factor (bFGF), 61.5 μg/plug recombinant ADAM 23 disintegrin domain (dd) expressed as a GST fusion protein (GST-ADAM23dd) (10X and 20X) and a glutathione control(GST) are shown.

### DETAILED DESCRIPTION OF THE INVENTION

Development of the nervous system involves an orderly set of connections between the various parts of the nervous system through the outgrowth of cellular protusions to create a functional network which is extremely complex. Axons and dendrites extend from the cell bodies by means of growth cones which travel along precisely specified paths to connect with a concrete target cell with which it is going to synapse. Neurons of different functional classes show distinctive surface characteristics that determine specific contact interactions with other cell surfaces, especially from glial cells, and with components of the extracellular matrix. Such interactions are of major importance for leading neuronal growth cones toward their targets along precisely specified routes.

The αvβ3 integrin is abundantly expressed in the radial glial cells during mouse development and has been proposed to play an important role in the facilitation of neuronal migration within central nervous system (Hirsch et al. Dev. Dyn. 1994 201:108-120). αvβ3 integrin has also been shown to be involved in the progression of melanoma and the induction of neovascularization by tumor cells (Seftor et al. Proc. Natl Acad. Sci. USA 1992 89:1557-1561; Brooks et al. Cell 1994 79:1157-1164). Further, the expression of αvβ3 integrin in undifferentiated neuroblastoma cells in vivo has been proposed to contribute to the rapid growth of these tumors and their tendency to metastasize (Gladson et al. Am. J. Pathol. 1996 148:1423-1434).

A member of the cellular disintegrin family, ADAM 23 has now been identified as interacting specifically with αvβ3 integrin. Further, this interaction is demonstrated herein to promote adhesion of cells of neural origin. It is believed that ADAM 23, through its disintegrin-like domain, functions as an adhesion molecule involved in αvβ3-mediated cell interactions occurring in normal and pathological processes. Expression of ADAM 23 and αvβ3 integrin has been detected in various tumor cell lines including tumors of neural origin melanoma, prostate and breast cancer cell lines. It is believed that the interaction of ADAM 23 and αvβ3 integrin leads to angiogenesis and the induction of active matrix metalloproteinases, ultimately leading to progression of malignant tumors.

The present invention relates to compositions including nucleic acid sequences and peptides, and vectors and host cells expressing the nucleic acid sequences and peptides for use in identifying and designing modulators of integrin-mediated cell-cell interactions relating to the interaction of ADAM 23 with αvβ3 integrin. The present invention also relates to methods of altering integrin-mediated cell-cell interactions through modulating the interaction of ADAM 23 with αvβ3 integrin. In one embodiment of the present invention, modulators of the interaction of ADAM 23 with αvβ3 integrin are used therapeutically to alter angiogenesis and induction of active matrix metalloproteinases facilitating migration of tumor cells, thereby inhibiting tumor progression. In another embodiment, modulators of the interaction of ADAM 23 with αvβ3 integrin are used therapeutically to alter neural tissue growth. For purposes of this invention by the term “modulate”, “modulating” and “modulation”, it is meant to up-regulate or induce interactions of ADAM 23 with αvβ3 integrin, to down-regulate or inhibit the interaction of ADAM 23 with αvβ3 integrin, or to block or interfere with the interaction of ADAM 23 with αvβ3 integrin. Thus, by “modulator” it is meant to be inclusive of agents which up-regulate or induce interactions of ADAM 23 with αvβ3 integrin, agents which down-regulate or inhibit the interaction of ADAM 23 with αvβ3 integrin, or agents which block or interfere with the interactions of ADAM 23 with αvβ3 integrin.

The full-length cDNA encoding ADAM 23, a member of the cellular disintegrin family was cloned. A nucleic acid sequence of ADAM 23 is depicted in SEQ ID NO:2. An amino acid sequence encoded thereby is depicted in SEQ ID NO:3. This new member of the ADAM family was first identified by screening the GenBank database of ESTs for sequences with similarities to those of previously described family members. Through this analysis, a 405 bp EST (R52569) was identified that, when translated, exhibited significant amino acid sequence similarity to the disintegrin domain characteristic of ADAMs. A cDNA containing part of this EST was generated by PCR amplification of DNA prepared from a human brain cDNA library and used as a probe to screen this library. Sequence analysis of one of the positive clones revealed an open reading frame coding for a protein of 832 amino acids with a predicted molecular mass of 91.9 kDa. An alignment of the deduced amino acid sequence revealed that this protein possesses all characteristic domains of the ADAM family members including propeptide, metalloproteinase-like, disintegrin-like and cysteine-rich domains, an EGF-like repeat, a transmembrane domain and a cytoplasmic tail. Further analysis of the identified amino acid sequence revealed that it shared similarities with a protein referred to as MDC3 which was cloned from a brain cDNA (Sagane et al. Biochem. J. (1998) 334:93-98). However, the cDNA of the present invention is approximately 1 kb longer than that of MDC3. Further, some sequence discrepancies have been identified.

Comparative analysis of the amino acid sequence encoded by the cDNA of the present invention revealed a significant similarity with other human ADAMs, the maximum percentage of identities being with ADAM 11 (53%) and ADAM 22 (51%). Following the nomenclature system for cellular disintegrins (see http://www.med.virginia.edu/˜jag6n/whitelab.html), this cDNA and the protein encoded thereby has been assigned the name ADAM 23.

Expression analysis of ADAM 23 in human tissues revealed a restricted pattern of expression to fetal and adult brain, muscle and lung. Tumor cells from neural origin such as NB 100, SH-S_{y}5_{y}, U373 and U87 MG also expressed this gene. ADAM 23 was also detected via RT-PCR analysis in human melanoma cell lines A375, Colo829, SKMel24 and HS695; murine melanoma cell lines B16F10 and M3(S91); human prostate carcinoma cell lines DU145, LNCap, and PC3; human breast carcinoma cell lines H3396, MCF7, MDA-MB231, and MDA-MB435; human umbilical vein endothelial cells (HUVEC) and weak band was detected in the human prostate carcinoma cell line MDA-PCa2b. These tumor cell lines also express αvβ3 integrin. Tumor cell lines from HL-60 (promyelocytic leukemia), K-562 (chronic myelogenous leukemia), Raji (Burkitt's lymphoma), HeLa (cervical adenocarcinoma), SW480 (colorectal adenocarcinoma), or A549 (lung adenocarcinoma) did not show significant levels of ADAM 23.

While ADAM 23 has a number of features characteristic of ADAM family members, its deduced amino acid sequence lacks essential residues conserved in metalloproteinases. This is indicative of the protein being involved in cell adhesion processes rather than in protease-mediated events. Experiments were therefore performed to elucidate the activities of ADAM 23 in cell-cell adhesion processes.

For these experiments, the predicted disintegrin domain of ADAM 23 was subcloned into the expression vector pGEX-3X, and the resulting plasmid, called pGEX-3X ADAM 23, as well as the original vector, were transformed into E. coli BL21(DE3)pLysS. Transformed bacteria were induced with IPTG and protein extracts analyzed by SDS-PAGE. Extracts from bacteria transformed with the recombinant plasmid contained a fusion protein of about 40 kDa, which was not present in the control extracts. The recombinant protein was purified by affinity chromatography in a glutathione-Sepharose 4B column, which was eluted with a reduced glutathione-containing buffer. After elution and SDS-PAGE analysis of proteins present in the chromatographic eluate, a single band of the expected size was detected.

The activity of the purified disintegrin domain of ADAM was then examined. Wells of microtiter plates were coated with the recombinant protein and seeded with NB100 human neuroblastoma cells. After rinsing the wells to remove unbound cells, bound cells were stained and quantified. It was found that ADAM 23-GST promoted cell adhesion in a similar manner to that observed when wells were coated with fibronectin. In contrast, wells coated with GST, albumin or buffer alone did not support any significant cell adhesion. Morphological studies of NB100 cells adherent to ADAM 23-GST or fibronectin using light and scanning electron microscopy revealed differences in cell morphology primarily related to changes in the number and length of surface protrusions. The structure of the actin cytoskeleton in NB100 cells adherent to either ADAM 23 or fibronectin was also examined. Neuroblastoma cells adherent to fibronectin showed a conventional F-actin distribution including relatively little F-actin in the central region of the cell and concentrated F-actin in a layer just beneath the plasma membrane. Cells adherent to ADAM 23 contained actin filaments mainly located at specific cortical regions. However, compared with cells adherent to fibronectin, these cells tended to have decreased levels of assembled actin filaments and a lower polarized pattern. In both, cells adherent to ADAM 23 and cells adherent to fibronectin, phalloidine labeling was not uniform, but usually was relatively dense in some areas and relatively sparse in others. Some of the dense labeling occurred in fairly distinct patches localized in close apposition to the plasma membrane. To confirm that these patches were actin-filament attachment sites in the plasma membrane and to study their distribution, staining of the same cells with antibodies to vinculin was performed. A clear relationship between the sites of vinculin localization, the actin-filament bundles and the sites of filopodial protrusion was observed. Although differences in the vinculin labeling pattern between cells adherent to either ADAM 23 or fibronectin were found, such differences were restricted to the degree of aggregation being higher in cells adherent to fibronectin. Nevertheless, in both cases, vinculin-positive patches were heterogeneously distributed, being concentrated at specific cortical regions which are believed to correspond to the leading edge of the cells.

Further analysis of the ADAM 23-promoted cell adhesiveness revealed that this effect was dose-dependent. In addition, the attachment of NB 100 neuroblastoma cells was stimulated in the presence of divalent cations like Mn²⁺ and Mg²⁺. Similar results were obtained when these experiments were performed with other cells from neural origin such as SH-S_{y}5_{y}, U373, and U87 MG. In contrast, when these experiments were performed with other cell lines from different sources including HT1080, HeLa, or T47D cells, no significant ADAM 23-mediated adhesion was observed. Accordingly, the effect of this cellular disintegrin on cell adhesion is dependent on the presence of specific integrins in the adherent cells.

The αvβ3-ADAM 23 interaction was examined by incubation of sepharose beads containing the ADAM 23 disintegrin domain fused to GST with purified αvβ3 integrin. After extensive washing to remove any unbound integrin, the presence of bound αvβ3 integrin was examined by SDS-PAGE of proteins solubilized in an SDS-containing buffer. Two bands corresponding to αv (145 kDa) and β3 (95 kDa) were detected in extracts from beads containing ADAM 23-GST but not in those derived from beads containing GST alone. The identity of these bands as αv and β3 was confirmed by Western blot analysis with antibodies raised against each integrin subunit. Similar experiments performed with other purified integrins such as α1β1 and α5β1, did not reveal any evidence of interaction with the recombinant ADAM 23. Antibodies blocking αvβ3 integrin function were able to reduce the ADAM 23 mediated adhesion of NB100 neuroblastoma cells, whereas a β1 blocking antibody did not show any significant effect on activity.

Additional experiments directed to analyze the interaction between αvβ3 integrin and ADAM 23 in the context of the full-length ADAM 23 protein were performed. For these experiments, the full-length cDNA for ADAM 23 containing a linker encoding the HA epitope at its 3′-end, was cloned into the eukaryotic expression vector pcDNA3. The resulting plasmid (pcDNA3-ADAM 23-HA) was transfected into HeLa cells and the ability of transfected cells to bind αvβ3 integrin was examined. Wells of microtiter plates coated with this integrin strongly supported cell adhesion of HeLa cells transfected with the ADAM 23 expression vector. In contrast, HeLa cells transfected with pcDNA3 alone did not support any significant cell adhesion. To provide additional evidence that ADAM 23 was located at the cell surface, a prerequisite for mediating the observed cell adhesion effect, HeLa cells transfected with pcDNA3-ADAM 23-HA were analyzed by immunofluorescence with a monoclonal antibody against the HA viral epitope. A clear fluorescent pattern surrounding transfected cells was visualized in a serial optical section obtained using the confocal microscope. In contrast, untransfected HeLa cells did not show any evidence of immunofluorescence signal at the cell surface. Taken together, these results are indicative of ADAM 23 being located at the cell surface and being able to promote αvβ3-mediated cell adhesion.

Analysis of the amino acid sequence of ADAM 23 shows the absence of any Arg-Gly-Asp (RGD) motif. This sequence has been found to be the major structural determinant supporting αvβ3-mediated interactions in different systems, including those involving metargidin, the only cellular disintegrin described to date containing an RGD motif (Krätzschmar et al. J. Biol. Chem. 1995 271:4593-4598; Herren et al. FASEB J. 1997 11:173-180; Zhang et al. J. Biol. Chem. 1998 273:7345-7350). Based upon comparison of the amino acid sequence of different human disintegrins around the putative region involved in integrin-binding, a short motif (AVNECDIT; SEQ ID NO:1) was selected as a candidate mediating the observed effect of ADAM 23 on cell adhesion. To determine whether this sequence is actually involved in the adhesive effect, the Glu residue of the central position to Ala was mutated. The disintegrin-like domain of the mutant protein, designated mutADAM 23, was expressed as a fusion protein with GST in accordance with procedures described herein for the wild-type disintegrin domain of ADAM 23. After affinity chromatography purification, the recombinant mutant protein was used for cell adhesion assays. The mutant ADAM 23 showed a significantly lower adhesion promoting activity of NB100cells than the effect observed when the wild-type ADAM 23 protein was used. Further, when wells of microtiter plates were coated with the mutant ADAM 23 and seeded with SH-S_{y}5_{y} neuroblastoma cells, the observed cell adhesion promoting effect was of about 40% compared to that obtained with the wild type protein.

To further examine the role of the sequence motif AVNECDIT (SEQ ID NO:1) in mediating the cell adhesion promoting properties of ADAM 23, a synthetic peptide with the amino acid sequence of this region (pep330) and a “scrambled” peptide DCVTNIAE (pep331; SEQ ID NO:4) with the same amino acid composition were prepared. NB100 cells were incubated separately with both peptides prior to be seeded on plates containing ADAM 23. A significant loss of adherent cells was detected with samples incubated with pep330. In contrast, this effect was not observed in samples incubated with the scrambled peptide derived from the same protein region. Thus, human ADAM 23 specifically interacts with αvβ3 integrin through a protein region whose amino acid sequence is AVNECDIT (SEQ ID NO:1), and therefore in an RGD-independent manner.

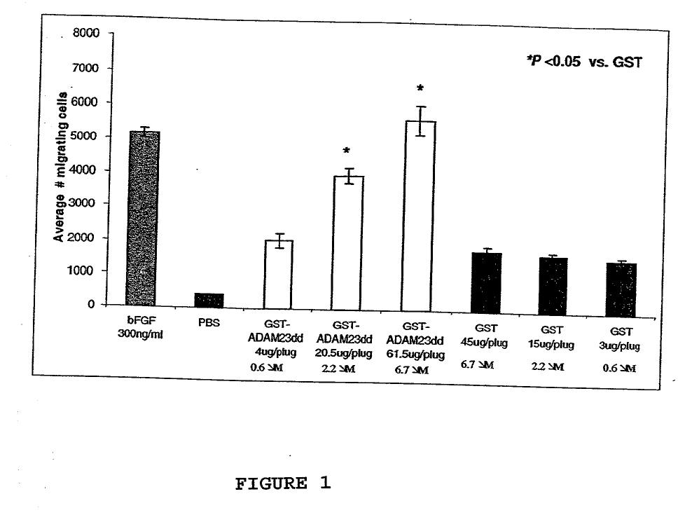

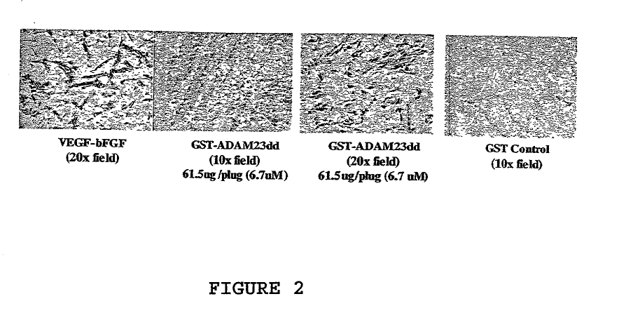

Recombinant ADAM 23 was also examined for its angiogenic activity in the tumor-independent MATRIGEL plug angiogenesis model. Typically, in this murine model a VEGF-bFGF mediated angiogenic response is shown by the migration of a large number of endothelial cells in representative MATRIGEL plug sections (VEGF-bFGF, FIG. 1). A similar angiogenic response is observed in plugs containing ADAM 23 (GST-ADAM23dd, FIG. 1) and this response is a dose-dependent (GST-ADAM23dd, FIG. 2). Interestingly, microscopic histologic analyses revealed that both endothelial and smooth muscle cells were present in plug sections containing ADAM 23. In contrast, a significant number of cells was not observed in plugs containing GST (FIGS. 1 and 2). These data demonstrate that the ADAM 23 disintegrin domain (containing the αvβ3 integrin binding motif, AVNECDIT (SEQ ID NO:1)) has angiogenic in vivo activity and this activity is similar to that observed with VEGF-bFGF or bFGF in this model.

The interaction of ADAM 23 with αvβ3 integrin is believed to be related to the biological and/or pathological functions of this disintegrin. These experiments demonstrating that ADAM 23 promotes adhesion of cells of neural origin, coupled with the predominant expression of ADAM 23 in the human brain in both fetal and adult stages, are indicative of ADAM 23 playing a role in the development and/or maintenance of neural functions. Further, the results reported herein for ADAM 23 and tumor cells are indicative of this cellular disintegrin having a role in tumor progression through the facilitation of integrin-mediated cell-cell interactions. Analysis of the nature of the signaling cascades initiated upon ADAM 23 binding to αvβ3 integrin are indicative of this interaction resulting in the induction of active matrix metalloproteinases, proteolytic enzymes believed to act as effector molecules modifying the surrounding of the involved cells and facilitating further migration of tumor cells. The interaction of ADAM 23 with αvβ3 integrin is also promotes angiogenesis as evidenced by the MATRIGEL plug assay.

Accordingly, the cDNA sequence for ADAM 23 depicted in SEQ ID NO:2 as well as vectors and host cells expressing the ADAM 23 protein or peptides thereof are useful in methods of identifying modulators of αvβ3-mediated cell interactions through altering the interaction of αvβ3 integrin with ADAM 23. Examples of peptides useful in these methods include peptides comprising the amino acid sequence AVNECDIT (SEQ ID NO:1) and fusion proteins such as the GST fusion protein comprising a peptide with amino acids 498-832 of the C-terminal portion of ADAM 23. In one embodiment, similar experiments to those conducted with pep330 and pep331 are performed with other potential modulators or test agents and changes in adherency of the cells upon contact with the test agent can be determined. High-throughput screening assays such as proximity-based assays can also be used. Preferably, the proximity based assay is a Scintillation Proximity Assay (SPA; Amersham Pharmacia Biotech.).

Modulators can also be identified in vitro using assays that employ recombinant protein reagents and/or cells expressing integrins. For example, recombinant integrin protein combinations, in particular αᵥβ₃ can be tested or their ability to bind to ADAM 23. These assays use specific antibodies and an enzyme-linked immunosorbent assay or ELISA to evaluate recombinant integrin proteins ability to bind ADAM 23-coated wells in the presence of a test agent.

Activity of test agents identified as modulators of the interaction of αvβ3 integrin and ADAM 23 in initial screening assays is then confirmed in secondary in vitro assays such as receptor/ligand binding assays with ADAM 23 and αvβ3 integrin; endothelial cell adhesion assays; melanoma cell adhesion assays; endothelial cell tube formation assays on MATRIGEL-coated plates; endothelial cell migration assays; and endothelial cell proliferation assays; and in vivo assays such as endothelial cell migration into subcutaneously implanted MATRIGEL plugs in athymic mice to evaluate angiogenic activity; and the Lewis lung carcinoma model. Inhibitors or antagonists of the interaction of αvβ3 integrin and ADAM 23 will decrease adherency and/or migration or progression of cells in these assays while agonists of this interaction will increase cell adherency and/or migration or progression of cells.

These methods can be used as screening assays for various test agents. Further, the knowledge that pep330 is an inhibitor of the interaction of αvβ3 integrin and ADAM 23 can be used in the rational design and selection of other inhibitors with similar structure and inhibitory activity. Accordingly, the present invention also relates to synthetic peptides comprising the amino acid sequence of AVNECDIT (SEQ ID NO:1) and variants thereof. By “variants” it is meant amino acid sequences with conservative amino acid substitutions which are also demonstrated to modulate the interaction of αvβ3 integrin and ADAM 23. For purposes of this peptide by “conservative amino acid substitutions” it is meant to include replacement, one for another, of the aliphatic amino acids such as Ala, Val, Leu and Ile, the hydroxyl residues Ser and Thr, the acidic residues Asp and Glu, and the amide residues Asn and Gln.

Modulators of the interaction of αvβ3 integrin and ADAM 23 are useful in altering integrin-mediated cell-cell interactions. Accordingly, the present invention relates to methods of altering integrin-mediated cell-cell interactions through use of modulators of the interaction of αvβ3 integrin and ADAM 23. Amounts of the modulator which are effective in altering integrin-mediated cell-cell interactions for incorporation into pharmaceutical compositions can be determined routinely by those of skill in the art in accordance with their pharmacological activities as determined by assays such as described herein. Compositions comprising modulators which inhibit or antagonize the interaction of αvβ3 integrin and ADAM 23 are expected to be useful in inhibiting angiogenesis and/or induction of active matrix metalloproteinases facilitating migration of tumor cells, both of which are involved in tumor progression. Thus, the present invention also provides methods of inhibiting angiogenesis and the induction of active matrix metalloproteinases facilitating migration of tumor cells via agents which inhibit the interaction of αvβ3 integrin and ADAM 23. Further, the present invention provides methods of inhibiting tumor progression through use of modulators which inhibit the interaction of αvβ3 integrin and ADAM 23. As discussed herein, high levels of expression of ADAM 23 and αvβ3 integrin are observed in tumors of neural origin, melanoma, breast carcinoma and prostate carcinoma. Accordingly, compositions and methods of the present invention are believed to be particularly useful in the inhibiting the progression of these types of tumors.

The involvement of ADAM 23 in αvβ3-mediated cell interactions occurring during neuronal growth is also indicative of agonists of the interaction of αvβ3 integrin and ADAM 23 being useful in promoting regeneration of normal neural tissue in neurodegenerative disorders and/or spinal cord injury. Accordingly, compositions comprising modulators which activate or agonize the interaction of αvβ3 integrin and ADAM 23 are expected to be useful in inducing growth of neural tissue.

Pharmaceutically acceptable vehicles useful in the present invention may comprise a carrier, adjuvant or vehicle that can be administered to a subject, incorporated into a composition of the present invention, and which do not destroy the pharmacologic activity thereof. Examples of pharmaceutical vehicles useful in the present invention include, but are not limited to, ion exchangers, alumina, aluminum stearate, lecithin, self-emulsifying drug delivery systems such as d(-tocopherol polyethyleneglycol 1000 succinate, surfactants used in pharmaceutical dosage forms such as TWEENS and other similar polymeric delivery matrices, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethocellulose, polyacrylates, waxes, polyethylene-polyoxypropylene-block polymers, and wool fat. Cyclodextrins such as α-, β- and γ-cyclodextrin, or chemically modified derivatives such as hydroxyalkylcyclodextrins, including 2- and 3-hydroxypropyl-β-cyclodextrins, or other solubilized derivatives can also be used to enhance delivery of the compositions of the present invention.

Various pharmaceutical formulations comprising compositions of the present invention can be prepared routinely by those of skill in the art using conventional solid or liquid vehicles or diluents, as well as pharmaceutical additives, selected in accordance with the desired mode of administration.

Compositions of the present invention can be administered by any suitable means, for example orally, such as in the form of tablets, capsules, granules or powders; sublingually; bucally; parenterally, such as by subcutaneous, intracutaneous, intravenous, intramuscular, intraarticular, intraarterial, intrasynovial, or intasternal, intrathecal, intralesional and intracranial injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic pharmaceutically acceptable vehicles. The compositions of the present invention can be administered in a form suitable for immediate release. Alternatively, an extended release formulation can also be used. Compositions of the present invention can also be administered liposomally.

Exemplary compositions for oral administration include: suspensions which may contain, for example, microcrystalline cellulose for impairing bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binder, extenders, disintegrants, diluents and lubricants known in the art. Compositions of the present invention can also be delivered sublingually or bucally through the oral cavity via, for example, molded tablets, compressed tablets or freeze-dried tablets. Examples of fast dissolving diluents for use in these formulations include, but are not limited to, mannitol, lactose, sucrose and/or cyclodextrins. Such formulations may further comprise high molecular weight excipients such as celluloses (avicel) or polyethylene glycol. Excipients to aid in mucosal adhesion such as hydroxypropylcellulose, hydroxypropylmethylcellulose, sodium carboxymethyl cellulose, maleic anhydride copolymer and agents to control release such as polyacrylic copolymer can also be incorporated into these formulations. In addition, the formulations may comprise lubricants, glidants, flavors, coloring agents and stabilizers which ease fabrication and use.

Exemplary compositions for nasal aerosol or inhalation administration include solutions in saline. These solutions may also contain preservatives such as benzyl alcohol, absorption promoters to enhance bioavailability and/or solubilizing or dispersing agents.

Exemplary compositions for parenteral administration include injectable solutions or suspensions which may contain, for example, suitable non-toxic parenterally acceptable diluents or solvents such as mannitol, 1,3-butanediol, water, Rhinger's solution, an isotonic sodium chloride solution, or other suitable dispersing or wetting and suspending agents including synthetic mono- or di-glycerides and fatty acids such as oleic acid.

Exemplary compositions for rectal administration include suppositories which may contain, for example, a suitable non-irritating excipient such as cocoa butter, synthetic glyceride esters or polyethylene glycols, which are solid at room temperature, but which liquefy and/or dissolve in the rectal cavity to release the active compound.

Exemplary compositions for topical administration include a topical carrier such as PLASTIBASE (mineral oil gelled with polyethylene)

The following nonlimiting examples are provided to further illustrate the present invention.

### EXAMPLES

### Example 1

### Materials

Restriction endonucleases and other reagents used for molecular cloning were purchased from Boehringer Mannheim (Mannheim, Germany). Double-stranded DNA probes were purchased from Amersham International (Buckinghamshire, UK) and were radiolabeled with [α-³²P]dCTP (3000 Ci/mmol) using a commercial random-priming kit from the same company. A human brain CDNA library constructed in λDR2 and Northern blots containing polyadenylated RNAs from different adult and fetal human tissues were purchased from Clontech (Palo Alto, Calif.). Synthetic peptides were obtained from the Molecular Biology Facilities Unit (University of Leicester, UK). Human neuroblastoma cells used in these experiments included NB100 and SH-S_{y}5_{y}. Astrocytoma cell lines used in these experiments included U373 and U87 MG. All media and supplements for cell culture were obtained from Sigma except for fetal calf serum, which was from Boehringer Mannheim.

### Example 2

### Isolation of a cDNA Clone for ADAM 23 from a Human Brain CDNA Library

A search of the GenBank database of human ESTs for sequences with homology to members of the ADAM family led to the identification of a sequence (R52569; WashU-Merck EST project) derived from a brain cDNA clone. To obtain this DNA fragment, PCR amplification of a human brain cDNA (Clontech) was performed with two specific primers 5′-CAACAAAGCTATTTGAGCCCACGG (SEQ ID NO:5) and 5′-TTGGTGGGCACTGACCAGAGTCT (SEQ ID NO:6), derived from the R52569 sequence. The PCR reaction was carried out in a GeneAmp 2400 PCR system from Perkin-Elmer/Cetus for 40 cycles of denaturation (94° C., 15 seconds), annealing (64° C., 20 seconds), and extension (72° C., 20 seconds). The 262 bp PCR product amplified from human brain cDNA, was cloned into a SmaI-cut pBluescript II SK vector, and its identity confirmed by nucleotide sequencing. This cDNA was then excised from the vector, radiolabeled and used to screen a human brain cDNA library in accordance with standard procedures as described by Maniatis et al. (Molecular Cloning: A Laboratory Manual. Cold Spring Harbor, N.Y. 1982).

### Example 3

### Northern blot Analysis

Northern blots containing poly(A)⁺ RNAs from different fetal and adult human tissues were obtained from Clontech (Palo Alto, Calif.). These blots were prehybridized at 42° C. for 3 hours in 50% formamide, 5× saline-sodium phosphate-EDTA, 2× Denhardt's solution, 0.1% SDS, and 100 μg/ml denatured herring sperm DNA, and then hybridized for 16 hours under the same conditions with the full-length cDNA isolated for ADAM 23. Filters were washed with 0.2× SSC and 0.1% SDS for 2 hours at 50° C. and subjected to autoradiography. RNA integrity and equal loading were assessed by hybridization with an actin probe as indicated by Clontech.

### Example 4

### RT-PCR Amplification

To assay the presence of ADAM 23 in neuroblastoma cell lines, total RNA was isolated from NB100 and SH-S_{y}5_{y} cells by guanidium thiocyanate-phenol-chloroform extraction, and used for cDNA synthesis with the RNA PCR kit from Perkin-Elmer. After reverse transcription (RT) using 1 μg total RNA and random hexamers as primers according to the instructions of the manufacturer, the whole mixture was used for PCR with the two specific oligonucleotides corresponding to the disintegrin domain of ADAM 23 as described in Example 2. Negative controls were performed using all reagents with the exception of the forward primer.

### Example 5

### Construction of an Expression Vector for ADAM 23 and Expression in Escherichia coli

A 975 bp fragment of the ADAM 23 CDNA containing the disintegrin-like domain, was generated by PCR amplification with primers 5′-TAGGGATCCCAAAGCTATTTGAGCCCA (SEQ ID NO:7) and 5′-ATGAAGATTTGGTGGGCA (SEQ ID NO:8). The PCR amplification was performed for 20 cycles of denaturation (95° C., 20 seconds), annealing (52° C., 20 seconds), and extension (68° C., 20 seconds), followed by 10 additional cycles of denaturation (95° C., 15 seconds), annealing (62° C., 15 seconds), and extension (68° C., 2 minutes) using the Expand Long PCR kit and the GeneAmp 9700 PCR system. Due to the design of the oligonucleotides, the amplified fragment could be cleaved at the 5′-end with HindIII and ligated in frame into the pGEX-3× E. coli expression vector (Invitrogen) previously cleaved with HindIII-SmaI. The expression vector was transformed into BL21(DE3)pLysS competent E. coli cells and grown on agar plates containing chloramphenicol and ampicillin. Single colonies were used to inoculate 2 ml cultures in 2YT medium supplemented with 33 μg/ml chloramphenicol and 50 μg/ml ampicillin. 500 μl of the corresponding culture was used to inoculate 200 ml of 2YT medium containing the above antibiotics. After culture reached an OD₆₀₀ of 0.6, expression was induced by addition of isopropyl-1-thio-β-D-galactopyranoside (IPTG) (0.5 mM final concentration) followed by further incubation for 3-20 hours at 30° C. Cells were collected by centrifugation, washed, and resuspended in 0.05 volumes of phosphate buffered saline, lysed via a French press, and centrifuged at 20,000× g for 20 minutes at 4° C. The soluble extract was incubated with glutathione-Sepharose 4B (Pharmacia) and eluted with glutathione elution buffer (10 mM reduced glutathione in 50 mM Tris-HCl, pH 8.0) following the manufacturer's instructions.

### Example 6

### Adhesion Assays

Cell adhesion assays were performed in accordance with procedures described by Luque et al. (FEBS Lett 1994 346:278-284). In these assays, 96-well immunoplates (MaxiSorp, Nunc, Denmark) were coated with 0.1 ml of PBS containing different amounts of BSA, glutathione S-transferase (GST), and ADAM 23/GST. After incubating 16 hours at 4° C., wells were blocked with DMEM containing 2.5% BSA, for 2 hours at 37° C. Then, NB100 neuroblastoma cells (approximately 50,000 cells per well) were added in Dulbecco's modified Eagle's medium (DMEM) supplemented with 1% BSA and incubated at 37° C. for 2 hours. For experiments directed to analyze the effect of divalent cations, the cells were washed three times in PBS, and resuspended in the same buffer supplemented either with 1 mM MgCl₂, 50 μM MnCl₂, 1 mM CaCl₂, or 1 mM MgCl₂ plus 5 mM EDTA. Non-bound cells were removed by rinsing the wells with serum-free medium, whereas bound cells were fixed with methanol and stained with Giemsa. Cells were counted per unit area with the aid of an inverted light microscope, using a 20× high powered objective and an ocular grid. For inhibition studies cells were pretreated for 30 minutes before the addition to the coated wells of mAb LM 609 (used at 1:400 dilution of ascites) or synthetic peptides (20 or 40 μg/ml) corresponding to the disintegrin loop of ADAM 23 (AVNEDCDIT, peptide 330 (SEQ ID NO:1)) or a “scrambled” peptide (DCVTNIAE, peptide 331 (SEQ ID NO:4)). In all cases, experimental treatments were performed in triplicate with a minimum of three areas counted per well.

### Example 7

### Scanning Electron Microscopy

Glass coverslips (12 mm diameter) were immersed in 60% HNO₃ for 1 hour, washed with distilled water, immersed in 7% NaOH and washed with water again. After drying, coverslips were placed in a 24-well tissue culture plate and coated with ADAM 23 or fibronectin in PBS (20 μg/ml). After overnight incubation at 4° C., coverslips were washed with PBS to remove free protein, and coated with 2.5% BSA. NB100 cells were then seeded (approximately 15,000 cells/cm²) in the same buffer used for cell adhesion experiments and allowed to adhere for 2 hours at 37° C. Unbound cells were then removed by washing with free serum medium and adherent cells were fixed with 2.5 glutaraldehyde in 0.1 M cacodylate buffer (pH 7.5) for 3 hours, and then washed, osmicated, dehydrated with acetone, critical point dried, and gold coated. Cells were then viewed under a Jeol JSM 6100 scanning electron microscope and photographed.

### Example 8

### Immunofluorescence Microscopy

NB100 cells were grown on glass coverslips as described in Example 7 and fixed with 3.7% paraformaldehyde in PBS for 20 minutes at room temperature and permeabilized with 0.2% Triton X-100 for 10 minutes. Coverslips were then incubated with 10% fetal bovine serum in PBS (30 minutes), followed by a 1:400 dilution of a commercial anti-vinculin monoclonal antibody (Sigma Co.) for 1 hour. After washing with PBS, incubation was made with a mix of a 1:500 dilution of a goat-antirabbit IgG FITC conjugated antibody (Amersham). For staining of filamentous actin, 0.1 μg/ml of rhodamine-phalloidine was included during incubation with the secondary antibodies. Finally, washed coverslips were mounted and cells were examined using a Zeiss fluorescent microscope equipped with a CCD camera (Photometrics).

### Example 9

### Construction of Eukaryotic Expression Vectors for ADAM 23-HA and Immunolocalization

A full-length cDNA encoding ADAM 23 was PCR amplified with oligonucleotides Ad23-D (5′-TATGAGCCATGAAGCCGCCCG-3′ (SEQ ID NO:9)) and Ad23-R (5′-GATGGGGCCTTGCTGAGTAGG-3′ (SEQ ID NO:10)), and cloned in the EcoRV site of a modified pcDNA3 vector containing a 24 bp linker coding for the hemagglutinin (HA) epitope of human influenza virus. Thus, the resulting ADAM 23 protein was HA-tagged at the COOH-terminus. HeLa cells were transfected with 1 μg of plasmid pcDNA3-ADAM 23-HA or pcDNA alone, using Lipofectamine reagent (Gibco-BRL), according to the manufacturer's instructions. Transfected cells were used for binding experiments to purified αvβ3 integrin or to protein extracts from integrin-transfected CHO cells as described in Example 9, with the exception that experiments were performed without divalent cations. For immunolocalization experiments, 48 hours after transfection, cells were fixed for 10 minuntes in cold 4% paraformaldehyde in PBS, washed in PBS, and incubated for 10 minutes in 0.2% Triton X-100 in PBS. Fluorescent detection was performed by incubating the slides with monoclonal antibody 12CA5 (Boehringer Mannheim) against HA (diluted 1:100), followed by another incubation with goat anti-mouse fluoresceinated antibody (diluted 1:50). Antibodies were diluted in blockage solution (15% fetal calf serum in PBS). After washing in PBS, slides were mounted with vectashield (Vector, Burlingame, Calif.) and observed in a BioRad confocal laser microscope.

### Example 10

### Site-directed Mutagenesis

The E466A mutation in the disintegrin loop of ADAM 23 was carried out by PCR-based methods. An oligonucleotide containing the mutation 5′-GTAATATCACACGCGTTCACAGCA (with G indicating a change in the original sequence from T to G (SEQ ID NO:11)), and a second oligonucleotide containing a BamHI site (5′-GTGGATCCCCAAGCTATTG (SEQ ID NO:12)) were first used to PCR amplify a DNA fragment. This amplified product was then used as a “megaprimer” for a second PCR amplification with an oligonucleotide corresponding to the 3′ end of the cloning site of pGEX-3X. PCR conditions were 94° C., 2 minutes (1 cycle), and 94° C., 0.1 seconds; 60° C., 0.1 seconds, 68° C., 30 seconds (20 cycles). The PCR product of the expected size was digested with BamHI and EcoRI and cloned in pGEX-3X. The presence of the mutation was confirmed by nucleotide sequencing. Finally, production of the recombinant mutant protein in Escherichia coli was carried out as described in Example 5.

### Example 11

### Western-blot Analysis

Purified integrins (0.3 g αvβ3, α1β1 or α5β1) (Chemicon International Inc., Temecula, Calif.) were incubated with Sepharose 4B beads containing 0.5 μg of disintegrin-GST, in a buffer containing 50 mM Tris-HCl, 200 mM NaCl and 0.2 mM MnCl₂ (pH 7.4), for 4 hours at 37° C. After incubation, beads were washed six times with 200 μl of the same buffer to remove unbound protein. Beads were then resuspended in Laemmli buffer and after boiling, solubilized proteins were loaded in a 6% SDS-PAGE gel, and visualized by silver staining. Alternatively, samples were blotted to a nitrocellulose membrane and the presence of αv or β3 integrin subunits was detected using polyclonal antibodies raised against these subunits. Similarly, the putative presence of β1 integrin subunits was examined with the B3B11 monoclonal antibody (Chemicon International Inc.). Western-blots were visualized by enhanced chemiluminescence according to the manufacturer's instructions (ECL, Amersham).

### Example 12

### Murine MATRIGEL Plug Angiogenesis Model

The angiogenic activity of ADAM 23 was evaluated in the murine MATRIGEL plug angiogenesis model. On day 0, ice-cold MATRIGEL (Becton-Dickinson, Bedford, Mass.) containing either vascular endothelial growth factor (VEGF, Pepro Tech, Inc., Rocky Hill, N.J.), basic fibroblast growth factor (bFGF, Pepro Tech, Inc., Rocky Hill, N.J. ), glutathione (GST), saline buffer (PBS) or recombinant ADAM 23 disintegrin domain (dd) expressed as a GST fusion protein (GST-ADAM23dd) was implanted subcutaneously in female athymic (BALB/c nu/nu; Harlan, Indianapolis, Ind.) mice. The MATRIGEL polymerized immediately after implantation into mice forming a gel plug. On day 7, individual MATRIGEL plugs were harvested, fixed in 10% buffered formalin, embedded in paraffin, sectioned, and stained with hematoxylin-eosin. The number of endothelial cells present in each plug section was measured using a video imaging system (Image Pro-Plus, Empire Imaging, Princeton, N.J.). Fifty fields (at 20×) per plug section were randomly counted. Data are presented as “Average Number of Migrating Cells” and the p-value was determined using a two-tailed Student T-Test.

What is claimed is:

1. An isolated nucleic acid sequence encoding ADAM 23.

2. The isolated nucleic acid sequence of claim 1 comprising SEQ ID NO:2.

3. A vector comprising the nucleic acid sequence of claim 1.

4. A host cell transfected with the vector of claim 3.

5. A method for identifying modulators of integrin-mediated cell-cell interactions comprising contacting a host cell expressing ADAM 23 or a peptide thereof with a test agent and determining the ability of the test agent to alter interaction of ADAM 23 or the peptide with αvβ3 integrin wherein a test agent which alters the interaction of ADAM 23 or the peptide with αvβ3 integrin is identified as a modulator of integrin-mediated cell-cell interactions.

6. A composition which alters integrin-mediated cell-cell interactions comprising an agent identified in accordance with claim 5.

7. A synthetic peptide comprising SEQ ID NO:1 or a variant thereof which modulates the interaction of ADAM 23 with αvβ3 integrin.

8. A host cell which expresses the peptide of claim 7.

9. A method of modulating integrin-mediated cell-cell interactions comprising contacting cells with a modulator which alters the interaction of ADAM 23 with αvβ3 integrin.

10. The method of claim 9 wherein the integrin-mediated cell-cell interaction modulated is angiogenesis.

11. The method of claim 9 wherein the integrin-mediated cell-cell interaction modulated is induction of active matrix metalloproteinases facilitating migration of tumor cells.

12. The method of claim 9 wherein the integrin-mediated cell-cell interaction modulated is growth of neural tissue.

13. A method for inhibiting tumor progression in a patient comprising administering to the patient a modulator of the interaction of ADAM 23 with αvβ3 integrin.

14. The method of claim 13 wherein the modulator inhibits or antagonizes the interaction of ADAM 23 with αvβ3 integrin.

15. A method for inducing growth of neural tissue comprising contacting neural tissue with a modulator of the interaction of ADAM 23 with αvβ3 integrin.

16. The method of claim 15 wherein the modulator activates or agonizes the interaction of ADAM 23 with αvβ3 integrin.

17. A composition which alters integrin-mediated cell-cell interactions comprising a modulator of the interaction of ADAM 23 with αvβ3 integrin.

18. A pharmaceutical composition for altering integrin-mediated cell-cell interactions comprising an effective amount of a modulator of the interaction of ADAM 23 with αvβ3 integrin and a pharmaceutically acceptable vehicle.

19. A composition which inhibits tumor progression comprising a modulator of the interaction of ADAM with αvβ3 integrin, wherein said modulator inhibits or antagonizes the interaction of ADAM with αvβ3 integrin.

20. A composition which induces growth of neural tissue comprising a modulator of the interaction of ADAM with αvβ3 integrin, wherein said modulator activates or agonizes the interaction of ADAM with αvβ3 integrin.


SEQUENCE LISTING (12 sequences)

SEQ ID NO: 1 - protein, 8 aa, Artificial Sequence
  Description of Artificial Sequence Synthetic
AVNECDIT

SEQ ID NO: 2 - DNA, 4043 bp, Homo sapiens
gaattccgggttttttacttagtagctcaagcaagttattgagtcttagttttctgctctatgaaatgggtacacccctaacctcaaaggaatattgtgaataaataataaaagattatacatgtgaaacacccataggtagctgctatatcacataactcaggcattagactttgggcagccccagattagacattcctgactttggagacatcactgcgtgggcagggatatggatacctgagacttggcttgtcagatagtggtgggcatgcacctcacaggtgatgccccatggtggcagagacagcattaggggattgacatattgcagaactcttctctaatggggaacagatgtctaataacctcctttctgggagttgcataggatccaaattacttggtagaatcacaatggcagcaaaggcatttgaagagtatggtatgagatttccgaccaattgttttatttaatttgagaaataaagatataaatcattctgtagttttttagatatttagagagatgggaaggagtctaagaactttctggattttctggttgacccttaggaaaagcatggttacatccttcaataattcaatcccctgctgctacttgagcacatcgcaatgaccagctccatcacaaatcagcacgtgaaacccagggtaccctgcctggaaatgtttgactgggggctcttttgaatgttttagacattatacccctttcctcctaaatgtttcagtgtatgtttttttaaatcaagtctttatagacctggactcctctgcgtcccgccccgggagtggctgcgaggctaggcgagccgggaaagggggcgccgcccagccccgagccccgcgccccgtgccccgagcccggagccccctgcccgccgcggcaccatgcgcgccgagccggcgtgaccggctccgcccgcggccgccccgcagctagcccggcgctctcgccggccacacggagcggcgcccgggagctatgagccatgaagccgcccggcagcagctcgcggcagccgcccctggcgggctgcagccttgccggcgcttcctgcggcccccaacgcggccccgccggctcggtgcctgccagcgccccggcccgcacgccgccctgccgcctgcttctcgtccttctcctgctgcctccgctcgccgcctcgtcccggccccgcgcctggggggctgctgcgcccagcgctccgcattggaatgaaactgcagaaaaaaatttgggagtcctggcagatgaagacaatacattgcaacagaatagcagcagtaatatcagttacagcaatgcaatgcagaaagaaatcacactgccttcaagactcatatattacatcaaccaagactcggaaagcccttatcacgttcttgacacaaaggcaagacaccagcaaaaacataataaggctgtccatctggcccaggcaagcttccagattgaagccttcggctccaaattcattcttgacctcatactgaacaatggtttgttgtcttctgattatgtggagattcactacgaaaatgggaaaccacagtactctaagggtggagagcactgttactaccatggaagcatcagaggcgtcaaagactccaaggtggctctgtcaacctgcaatggacttcatggcatgtttgaagatgataccttcgtgtatatgatagagccactagagctggttcatgatgagaaaagcacaggtcgaccacatataatccagaaaaccttggcaggacagtattctaagcaaatgaagaatctcactatggaaagaggtgaccagtggccctttctctctgaattacagtggttgaaaagaaggaagagagcagtgaatccatcacgtggtatatttgaagaaatgaaatatttggaacttatgattgttaatgatcacaaaacgtataagaagcatcgctcttctcatgcacataccaacaactttgcaaagtccgtggtcaaccttgtggattctatttacaaggagcagctcaacaccagggttgtcctggtggctgtagagacctggactgagaaggatcagattgacatcaccaccaaccctgtgcagatgctccatgagttctcaaaataccggcagcgcattaagcagcatgctgatgctgtgcacctcatctcgcgggtgacatttcactataagagaagcagtctgagttactttggaggtgtctgttctcgcacaagaggagttggtgtgaatgagtatggtcttccaatggcagtggcacaagtattatcgcagagcctggctcaaaaccttggaatccaatgggaaccttctagcagaaagccaaaatgtgactgcacagaatcctggggtggctgcatcatggaggaaacaggggtgtcccattctcgaaaattttcaaagtgcagcattttggagtatagagactttttacagagaggaggtggagcctgccttttcaacaggccaacaaagctatttgagcccacggaatgtggaaatggatacgtggaagctggggaggagtgtgattgtggttttcatgtggaatgctatggattatgctgtaagaaatgttccctctccaacggggctcactgcagcgacgggccctgctgtaacaatacctcatgtctttttcagccacgagggtatgaatgccgggatgctgtgaacgagtgtgatattactgaatattgtactggagactctggtcagtgcccaccaaatcttcataagcaagacggatatgcatgcaatcaaaatcagggccgctgctacaatggcgagtgcaagaccagagacaaccagtgtcagtacatctggggaacaaaggctgcagggtctgacaagttctgctatgaaaagctgaatacagaaggcactgagaagggaaactgcgggaaggatggagaccggtggattcagtgcagcaaacatgatgtgttctgtggattcttactctgtaccaatcttactcgagctccacgtattggtcaacttcagggtgagatcattccaacttccttctaccatcaaggccgggtgattgactgcagtggtgcccatgtagttttagatgatgatacggatgtgggctatgtagaagatggaacgccatgtggcccgtctatgatgtgtttagatcggaagtgcctacaaattcaagccctaaatatgagcagctgtccactcgattccaagggtaaagtctgttcgggccatggggtgtgtagtaatgaagccacctgcatttgtgatttcacctgggcagggacagattgcagtatccgggatccagttaggaaccttcacccccccaaggatgaaggacccaagggtcctagtgccaccaatctcataataggctccatcgctggtgccatcctggtagcagctattgtccttgggggcacaggctggggatttaaaaatgtcaagaagagaaggttcgatcctactcagcaaggccccatctgaatcagctgcgctggatggacaccgccttgcactgttggattctgggtatgacatactcgcagcagtgttactggaactattaagtttgtaaacaaaacctttgggtggtaatgactacggagctaaagttggggtgacaaggatggggtaaaagaaaactgtctcttttggaaataatgtcaaagaacacctttcaccacctgtcagtaaacgggggagggggcaaaagaccatgctataaaaagaactgttccagaatcttttttttccctaatggacgaaggaacaacacacacacaaaaattaaatgcaataaaggaatcattaaaaaaaatagtaaatgattttttttccctcagcctgctggcacttaatatcttctaaatgatttggcatgatttttttttctttactaccgatgacaaactccagtggcatgaagatctaattttcaaaagggtaaaaactgcatggcatatatacaacaagctagcaagccaattctcagcaaaacctgcaacagaattc

SEQ ID NO: 3 - protein, 832 aa, Homo sapiens
MKPPGSSSRQPPLAGCSLAGASCGPQRGPAGSVPASAPARTPPCRLLLVLLLLPPLAASSRPRAWGAAAPSAPHWNETAEKNLGVLADEDNTLQQNSSSNISYSNAMQKEITLPSRLIYYINQDSESPYHVLDTKARHQQKHNKAVHLAQASFQIEAFGSKFILDLILNNGLLSSDYVEIHYENGKPQYSKGGEHCYYHGSIRGVKDSKVALSTCNGLHGMFEDDTFVYMIEPLELVHDEKSTGRPHIIQKTLAGQYSKQMKNLTMERGDQWPFLSELQWLKRRKRAVNPSRGIFEEMKYLELMIVNDHKTYKKHRSSHAHTNNFAKSVVNLVDSIYKEQLNTRVVLVAVETWTEKDQIDITTNPVQMLHEFSKYRQRIKQHADAVHLISRVTFHYKRSSLSYFGGVCSRTRGVGVNEYGLPMAVAQVLSQSLAQNLGIQWEPSSRKPKCDCTESWGGCIMEETGVSHSRKFSKCSILEYRDFLQRGGGACLFNRPTKLFEPTECGNGYVEAGEECDCGFHVECYGLCCKKCSLSNGAHCSDGPCCNNTSCLFQPRGYECRDAVNECDITEYCTGDSGQCPPNLHKQDGYACNQNQGRCYNGECKTRDNQCQYIWGTKAAGSDKFCYEKLNTEGTEKGNCGKDGDRWIQCSKHDVFCGFLLCTNLTRAPRIGQLQGEIIPTSFYHQGRVIDCSGAHVVLDDDTDVGYVEDGTPCGPSMMCLDRKCLQIQALNMSSCPLDSKGKVCSGHGVCSNEATCICDFTWAGTDCSIRDPVRNLHPPKDEGPKGPSATNLIIGSIAGAILVAAIVLGGTGWGFKNVKKRRFDPTQQGPI

SEQ ID NO: 4 - protein, 8 aa, Artificial Sequence
  Description of Artificial Sequence Synthetic
DCVTNIAE

SEQ ID NO: 5 - DNA, 24 bp, Artificial Sequence
  Description of Artificial Sequence Synthetic
caacaaagctatttgagcccacgg

SEQ ID NO: 6 - DNA, 23 bp, Artificial Sequence
  Description of Artificial Sequence Synthetic
ttggtgggcactgaccagagtct

SEQ ID NO: 7 - DNA, 27 bp, Artificial Sequence
  Description of Artificial Sequence Synthetic
tagggatcccaaagctatttgagccca

SEQ ID NO: 8 - DNA, 18 bp, Artificial Sequence
  Description of Artificial Sequence Synthetic
atgaagatttggtgggca

SEQ ID NO: 9 - DNA, 21 bp, Artificial Sequence
  Description of Artificial Sequence Synthetic
tatgagccatgaagccgcccg

SEQ ID NO: 10 - DNA, 21 bp, Artificial Sequence
  Description of Artificial Sequence Synthetic
gatggggccttgctgagtagg

SEQ ID NO: 11 - DNA, 24 bp, Artificial Sequence
  Description of Artificial Sequence Synthetic
gtaatatcacacgcgttcacagca

SEQ ID NO: 12 - DNA, 19 bp, Artificial Sequence
  Description of Artificial Sequence Synthetic
gtggatccccaagctattg